(19) United States
(12) Patent Application Publication
(10) Pub. No.: US 2025/0032641 A1
(43) Pub. Date: Jan. 30, 2025

(54) IMMUNE- STIMULATING COMPOUNDS LINKED TO GLYCOGEN-BASED POLYSACCHARIDE NANOPARTICLES FOR SENSITIZING CANCER CELLS TO A CHEMOTHERAPEUTIC DRUG

(71) Applicant: GLYSANTIS BIOTECH INC., Moffat (CA)

(72) Inventors: Jondavid DE JONG, Guelph (CA); Sheetij GHOSHAL, Mississauga (CA); Sarah POYNTER, Cambridge (CA)

(21) Appl. No.: 18/269,942

(22) PCT Filed: Dec. 24, 2021
(86) PCT No.: PCT/CA2021/051890

§ 371 Date: Jun. 27, 2023

Related U.S. Application Data
(60) Provisional application No. 63/130,944, filed on Dec. 28, 2020.

(51) Int. Cl. A61K 47/69; A61K 31/282; A61K 31/7076

(52) U.S. Cl. CPC A61K 47/6939; A61K 31/282; A61K 31/7076

(57) ABSTRACT

A method of sensitizing a cancer cell to a chemotherapeutic drug includes contacting the cell with an immune-stimulating compound covalently or non-covalently linked to glycogen-based polysaccharide nanoparticles having a molecular weight of 10⁶ to 10⁷ Da comprising α-D glucose chains, having an average 5 chain length of 11-12, with 1→4 linkage and branching point occurring at 1→6 and with a branching degree of between 6% and 13%.

### CROSS REFERENCE TO RELATED APPLICATIONS

This application is a national stage entry of PCT/CA2021/051890 filed Dec. 24, 2021, which claims priority to U.S. application No. 63/130,944 filed Dec. 28, 2020.

### FIELD OF THE INVENTION

The present invention relates to compounds for sensitizing cancer cells to chemotherapy treatments.

### BACKGROUND OF THE INVENTION

Chemoresistance within cancer presents a major obstacle within clinical settings. Broadly speaking chemoresistance can be either intrinsic or acquired. Intrinsic chemoresistance represents a situation whereby cancer cells are inherently resistant to therapy, whereas acquired resistance emerges during/after treatment. Every cancer has different response rates to chemotherapies, however a commonality across all cancers is eventual recurrence within a percentage of the treated population (ranges from low single digit percentages to 100%). Chemoresistance represents a major challenge in primary and secondary tumours across all tumour types. As an example, approximately 80% of ovarian cancer patients who have responded favourably to standard chemotherapy (platinum-based drugs in combination with paclitaxel), will develop recurring tumours resistant to platinum-based chemotherapy. This highlights the urgent need to develop strategies to either 1) decrease the EC50 of chemotherapies in initial treatment to decrease selection pressure and mitigate inherent resistance and/or 2) overcome acquired chemoresistance through re-sensitizing chemoresistant tumours.

SUMMARY OF INVENTION

Embodiments of the present invention include:
1. A method of sensitizing a cancer cell to a chemotherapeutic drug comprising contacting the cell with an immune-stimulating compound covalently or non-covalently linked to glycogen-based polysaccharide nanoparticles having a molecular weight of 10⁶ to 10⁷ Da comprising α-D glucose chains, having an average chain length of 11-12, with 1→4 linkage and branching point occurring at 1→6 and with a branching degree of between 6% and 13%.
2. The method of embodiment 1, wherein sensitizing a cancer cell to a chemotherapeutic drug comprises stimulating an immune response in the cancer cell, which comprises increasing expression of one or more gene(s) associated with the innate immune response, apoptosis and/or autophagy.
3. The method of embodiment 2, wherein stimulating an immune response comprises increasing expression of one or more of IFN-beta, ISG-15 and CXCL10.
4. The method of any one of embodiments 1 to 3, wherein sensitizing cancer cells to a chemotherapeutic drug comprises lowering the EC50 by >10%, >20%, >30%, >40%, >50%, >60%, >70%, >80%, or >90%.
5. The method of any one of embodiments 1 to 4, wherein the chemotherapeutic drug comprises a platinum-based compound, an anthracycline (including doxorubicin, daunorubicin, epirubicin, idarubicin), etoposide, 2′, 2′-difluoro 2′deoxycytidine (dFdC, gemcitabine), capecitabine, fluorouracil, a taxane (including Paclitaxel, Docetaxel), irinotecan, methotrexate, pemetrexed, topotecan, vinblastine, vincristine, auristatin E, auristatin F, dolastatin, maytansinol, monomethyl auristatin E, monomethyl auristatin F, monomethyl auristatin D, DM1, DM4, Pyrrolobenzodiazepine, alpha-aminitin, calicheamicin, campathecin, and/or mitomycin C.
6. The method of embodiment 5, wherein the chemotherapeutic drug comprises a platinum-based chemotherapeutic compound, preferably selected from carboplatin, cisplatin and oxaliplatin.
7. The method of embodiment 1, wherein sensitizing the cancer cell to a chemotherapeutic drug comprises inducing expression of PDL-1 in the cancer cell and wherein the chemotherapeutic drug comprises a checkpoint inhibitor.
8. The method of any one of embodiments 1 to 7, wherein the immune-stimulating compound is a TLR agonist, preferably selected from: double-stranded RNA, double-stranded DNA and single-stranded RNA, single-stranded DNA, or any synthetic analogs thereof including poly IC, CpG oligodeoxynucleotide (ODN), locked nucleic acid (LNA).
9. The method of embodiment 8, wherein the immune-stimulating compound is poly IC.
10. The method of any one of embodiments 1 to 9 wherein the immune-stimulating compound is covalently linked to the nanoparticles through a linking group.
11. The method of any one of embodiments 1 to 9 wherein the nanoparticles are cationic and the immune-stimulating compound is non-covalently linked to the nanoparticles through electrostatic interactions.
12. The method of any one of embodiments 1 to 11, wherein the immune-stimulating compound comprises between % 50-% 600 by weight relative to the glycogen-based polysaccharide nanoparticles.
13. The method of any one of embodiments 1 to 11 wherein the glycogen-based polysaccharide nanoparticles and the immune-stimulating compound are used in a weight ratio of 2:1.
14. The method of any one of embodiments 1 to 13, wherein the glycogen-based polysaccharide nanoparticles have a polydispersity index as measured by DLS of less than 0.3 and the glycogen-based polysaccharide nanoparticles linked to the immune-stimulating compound have an average particle diameter of between about 10 nm and about 300 nm.
15. The method of any one of embodiments 1 to 14 comprising contacting the cell with the chemotherapeutic drug within ±7 days, within ±48 hours, within ±24 hours or concurrently with contacting the cell with the immune-stimulating compound covalently or non-covalently linked to glycogen-based polysaccharide nanoparticles.
16. The method of any one of embodiments 1 to 15 wherein the cell is in a cancer patient and the method comprises administering a therapeutically effective amount of a pharmaceutical composition comprising the immune-stimulating compound covalently or non-covalently linked to the glycogen-based polysaccharide nanoparticles to the cancer patient.
17. The method of embodiment 16, wherein the nanoparticles are further covalently linked to one or more small molecules, proteins, peptides, antibodies or antibody fragments, aptamers, receptor ligands, for directing the nanoparticles to a specific cell type or cellular compartment.
18. The method of any one of embodiments 16 to 17, comprising administering the immune-stimulating compound covalently or non-covalently linked to glycogen-based polysaccharide nanoparticles within 7 days, preferably within 48 hours of administering the chemotherapeutic drug.
19. The method of any one of embodiments 16 to 17, comprising administering the immune-stimulating compound covalently or non-covalently linked to glycogen-based polysaccharide nanoparticles concurrently with the a chemotherapeutic drug.
20. The method of any one of embodiments 16 to 19, wherein the patient is diagnosed with glioma, thyroid cancer, lung cancer, colorectal cancer, stomach cancer, liver cancer, carcinoid, pancreatic, renal, urothelial, prostate, breast, cervical, endometrial, ovarian, melanoma, multiple myeloma, acute myeloid leukemia, indolent non-Hodgkin's lymphoma.
21. The method of any one of embodiments 16 to 20, wherein the patient is diagnosed with ovarian cancer or breast cancer.

### BRIEF DESCRIPTION OF FIGURES

FIG. 1 shows the ability of cationized phytoglycogen nanoparticles (Cat-PhG) to bind polyinosinic:polycytidylic acid (pI:pC) as demonstrated by electrophoretic mobility shift assay (EMSA). Three (3) micrograms of pI:pC was mixed with 2-fold dilution series of Cat-PhG harbouring a 0.88 degree of substitution (ds). After a 20-minute incubation at room temperature, samples were separated on a 1% agarose gel, stained with ethidium bromide, and subsequently imaged under UV light. Shown are the EMSA gels for Cat-PhG-0.88 demonstrating maximum loading ˜6:1 (poly:IC:PhG w/w).

FIG. 2 shows transcript levels of interferon-stimulated genes CXCL10 and ISG15, as well as immune checkpoint inhibitor target PDL1 in ovarian cancer cells (SKOV-3) after treatment with low molecular weight (LMW) and high molecular weight (HMW) pI:pC alone, or LMW and HMW pI:pC complexed with phytoglycogen nanoparticles. SKOV-3 cells were treated with media alone (CTRL), 125 ng/ml of pI:pC, or 125 ng/mL of poly IC complexed with 250 ng/ml of PhG (poly IC:PhG) for 6 hours. Transcript levels were measured using qRT-PCR.

FIG. 3 shows transcript levels of interferon-stimulated genes CXCL10 and ISG15, as well as immune checkpoint inhibitor target PDL1, in breast cancer cells (SKRBR-3) after treatment with LMW and HMW pI:pC alone, or LMW and HMW pI:pC complexed with phytoglycogen nanoparticles. SKRBR-3 cells were treated with media alone (CTRL), 2.5 μg/mL of pI:pC, or 2.5 μg/mL of poly IC complexed with 5 μg/mL of PhG (poly IC:PhG) for 6 hours. Transcript levels were measured using qRT-PCR.

FIG. 4 shows transcript levels of interferon beta (IFNB) and interferon-stimulated gene CXCL10 in breast cancer cells (MCF-7) after treatment with LMW and HMW pI:pC alone, or LMW and HMW pI:pC complexed with phytoglycogen nanoparticles. MCF-7 cells were treated with media alone (CTRL), 5 μg/mL of pI:pC, or 5 μg/mL of poly IC complexed with 10 μg/mL of PhG (poly IC:PhG) for 6 hours. Transcript levels were measured using qRT-PCR.

FIG. 5 shows transcript levels of interferon beta (IFNB) and interferon-stimulated stimulated genes CXCL10 and ISG15 in cervical cancer cells (HeLa) after treatment with HMW pI:pC alone, or LMW and HMW pI:pC complexed with phytoglycogen nanoparticles. HeLa cells were treated with media alone (CTRL), 5 μg/mL of pI:pC, or 5 μg/mL of poly IC complexed with 10 μg/mL of PhG (poly IC:PhG) for 6 hours. Transcript levels were measured using qRT-PCR.

FIG. 6 shows transcript levels of interferon-stimulated stimulated genes CXCL10, ISG-15,and immune checkpoint inhibitor target PDL1 in murine melanoma cells (B16F10) after treatment with LMW and HMW pI:pC alone, or LMW and HMW pI:pC complexed with phytoglycogen nanoparticles. Hela cells were treated with media alone (CTRL), 5 μg/mL of pI:pC, or 5 μg/mL of poly IC complexed with 10 μg/mL of PhG (poly IC:PhG) for 6 hours. Transcript levels were measured using qRT-PCR.

FIG. 7 shows cell viability as measured by alamar blue of pI:pC resistant SKOV-3 cells treated with carboplatin alone, or in combination with “free” pI:pC or glycogen nanoparticle bound pI:pC. Twenty-four (24) hours post seeding, cells were left untreated, or treated with EC50 carboplatin, 125 ng/ml pI:pC, 125 ng/ml pI:pC ionically bound to glycogen nanoparticles (1:1 w/w), or the combination of EC50 carboplatin and 125 ng/ml pI:pC, or EC50 carboplatin and 125 ng/ml nanoparticle bound pI:pC, and cell viability was measured after 72 hours incubation.

FIG. 8 shows cell viability as measured by alamar blue of ID8 murine ovarian cancer cells treated with carboplatin alone, or in combination with “free” pI:pC or glycogen nanoparticle bound pI:pC. Twenty-four (24) hours post seeding, cells were left untreated, or treated with 75 μM carboplatin, 5 μg/ml pI:pC, 5 μg/ml nanoparticle bound pI:pC (2:1 w/w), or the combination of 75 μM carboplatin and 5 μg/ml pI:pC, or 75 μM carboplatin and 5 μg/ml nanoparticle bound pI:pC, and cell viability was measured after 72 hours incubation.

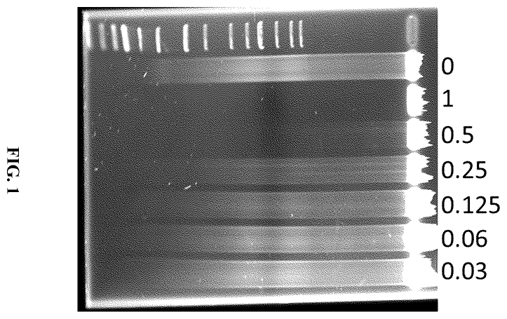

### DETAILED DESCRIPTION

Stimulating an immune response within a tumour presents a significant challenge to current cancer immune-therapies, as cancer cells are known to have defects within innate immune signaling pathways [1]. As demonstrated in the Examples, the inventors have found that pI:pC (also referred to herein as “Poly IC”) bound to glycogen nanoparticles surprisingly induces a robust innate immune response at doses where unbound pI:pC is ineffective. Even more surprisingly this trend is maintained across a number a cancer cell types (Breast, Ovarian, Cervical, Melanoma). The enhanced immune signalling would be expected to extend to any cancer with intact double-stranded RNA sensors (including TLR3, RIG-I, MDA5, PKR, DDX family of helicases).

Cancer cells can be divided into 2 categories, pI:pC responsive, and pI:pC non-responsive cells, with only pI:pC responsive cells demonstrating any significant benefit from pI:pC chemo sensitization when pI:pC is administered in its “free form” [2].

A method of sensitizing cancer cells to a chemotherapeutic drug (including cancer cells that are non-responsive to pI:pC in its free form) comprises contacting the cell with an immune-stimulating compound covalently or non-covalently linked to glycogen-based polysaccharide nanoparticles.

In one embodiment, normally refractive pI:pC resistant cancer cells are rendered susceptible to pI:pC-induced chemosensitivity.

As used herein, “a chemotherapeutic drug” refers to any chemical compound useful in the treatment of cancer. In some embodiments, a chemotherapeutic drug comprises a compound that inhibits mitosis (cell division) and/or induces DNA damage in a cell.

As used herein, “sensitizing cancer cells to a chemotherapeutic drug” refers to increasing the efficacy of the drug: in some embodiments, improving the efficacy of the drug in inhibiting cell division or inducing or enhancing cell apoptosis. In some embodiments, “sensitizing cancer cells to a chemotherapeutic drug” refers to lowering the dose of the compound required to be pharmaceutically effective (in some embodiments, inhibit cell division or induce or enhance cell apoptosis). In various embodiment, sensitizing cancer cells to chemotherapy comprises lowering the EC50 by >10%, >20%, >30%, >40%, >50%, >60%, >70%, >80%, or >90%.

As used herein, “stimulating an immune response” or grammatical variations thereof refers to inducing or enhancing an immune response, which comprises upregulating gene(s), or activating proteins, associated with the innate immune response, apoptosis and/or autophagy (examples of which are ISG15,CXCL10, interferon beta, caspase 9, caspase 3, caspase 7, BCL-2 protein family, Beclin 1, Atg5, Atg7, LC3, Atg 12 and Atg 16L1). In one embodiment, inducing or enhancing an immune response in a cancer cell includes increasing expression of one or more interferon-stimulated genes (ISGs). In one embodiment, inducing or enhancing an immune response comprises increasing expression of one or more of IFN-beta, ISG-15 or CXCL10.

Surprisingly, when tested in various cancer cells, the compositions provided herein induced immune-stimulating cytokines and chemokines at concentrations wherein the unbound immune-stimulating compound alone was ineffective.

In a further embodiment, sensitizing cancer cells to a chemotherapeutic drug comprises inducing expression of Programmed death ligand-1 (PDL1) in the cancer cells. Surprisingly, when tested in various cancers cells which demonstrate no baseline PDL1 expression, compositions provided herein induced robust expression of PDL1 when compared to the immune-stimulating compound alone.

Programmed death ligand-1 (PDL1) is expressed on various cancer and immune cells. PDL1 upregulation is believed to play an important role in evasion of the immune system by cancer cells via binding of programmed death-1 (PD-1) on T-lymphocytes. High tumor expression of PDL1 has been associated with tumor aggression, but has also been associated with an improved response to anti-PDL1 therapy (e.g. PDL1 checkpoint inhibitors). Currently, response rates to PDL1 check point inhibitors (CPIs) ranges from 10-15% across all cancer types. Concomitant generation of immune signaling molecules such as CXCL10, and PDL1 surface expression would render non-responsive tumours, responsive to PDL1 CPIs. In one embodiment, the chemotherapeutic compound is a CPI.

As demonstrated in the Examples, the inventors have found that pI:pC bound to glycogen nanoparticles, surprisingly, sensitizes pI:pC resistant cells to a chemotherapeutic drug, at levels where free pI:pC is ineffective. Thus in some embodiments, methods of the present invention are applied to cancer cells that do not exhibit any observable expression of PDL1 when contacted with the same dose of the immune-stimulating compound that is not covalently or non-covalently linked to glycogen-based polysaccharide nanoparticles as taught herein.

pI:pC chemotherapy sensitization is linked to increased apoptosis and autophagy signaling, and TLR3 stimulation results in down regulation of drug efflux pumps P-gp and MRP-1 [2, 3], thus the chemo-sensitization observed by the inventors may extend to any chemotherapeutic drug (i.e. a chemical compound useful in treatment of cancer) that induces apoptosis, autophagy, or is transported by P-gp and/or MRP-1. As demonstrated in the Examples, the inventive compositions and methods as provided herein upregulate both immune stimulating cytokines and chemokines, as well as PDL1 expression in cancer cells at levels where free pI:pC is ineffective. In some embodiments, methods of the present invention are applied to cancer cells to generate alterations within cancer cell innate immune signal transduction pathways and surface expression of PDL1, which renders the cells more sensitive to the combination of cell-based therapies (CAR-T, NK etc) and CPIs.

Such chemotherapies include, but are not limited to: platinum-based chemotherapeutic agents, anthracyclines (including doxorubicin, daunorubicin, epirubicin, idarubicin), etoposide, 2′, 2′-difluoro 2′deoxycytidine (including dFdC, gemcitabine), capecitabine, fluorouracil, taxanes (including Paclitaxel, Docetaxel), irinotecan, methotrexate, pemetrexed, topotecan, vinblastine, vincristine, auristatin E, auristatin F, dolastatin, maytansinol, monomethyl auristatin E, monomethyl auristatin F, monomethyl auristatin D, DM1, DM4, Pyrrolobenzodiazepine, alpha-aminitin, calicheamicin, and/or campathecinmitomycin C.

In one embodiment, the a chemotherapeutic drug is a platinum-based chemotherapeutic compound, preferably selected from carboplatin, cisplatin and oxaliplatin.

The chemotherapeutic compound may be used in its free drug form, bound or encapsulated within a nanoparticle, bound to a targeting moicty (for example an antibody, antibody drug conjugate.)

In one embodiment, the chemotherapeutic compound and immune-stimulating compound are both covalently or non-covalently linked to the same glycogen-based polysaccharide nanoparticle. In one embodiment, the chemotherapeutic compound can be covalently linked to the polysaccharide nanoparticle and the immune-stimulating compound may be non-covalently linked to the polysaccharide nanoparticle. In one embodiment, both the chemotherapeutic compound and immune-stimulating compound may be covalently linked to the polysaccharide nanoparticle.

TLR3, one of several receptors for pI:pC, is the critical receptor for chemo-sensitization [2]. In one embodiment, the methods and compositions as described herein are for use in the treatment of cancers that express TLR3; in various embodiments, glioma, thyroid cancer, lung cancer, colorectal cancer, stomach cancer, liver cancer, carcinoid, pancreatic, renal, urothelial, prostate, breast, cervical, endometrial, ovarian, melanoma, multiple myeloma, acute myeloid leukemia, indolent non-Hodgkin's lymphoma.

Glycogen and phytoglycogen are composed of molecules of α-D glucose chains having an average chain length of 11-12, with 1→4 linkage and branching point occurring at 1→6 and with a branching degree of about 6% to about 13%. Glycogen and phytoglycogen molecules may be modified as described further below; “glycogen-based polysaccharide” refers to a polysaccharide exhibiting this structure although subject to further modifications.

In one embodiment, the formulations comprise a carbohydrate-based nanoparticle and an immune-stimulating compound. In one embodiment the carbohydrate-based nanoparticles are glycogen-based nanoparticles. In various embodiments, the glycogen-based nanoparticles may be naturally derived (i.e. produced using as a starting material a natural source of glycogen or phytoglycogen), or chemically synthesized, unmodified, or chemically modified to carry altered physiochemical characteristics (positive or negative charge, hydrophobicity, or combination thereof) or targeting moieties.

In one embodiment, the immune-stimulating compound is directly covalently linked to the glycogen nanoparticle or covalently linked via a linker, for instance an amino group or a carboxy group.

In another embodiment, the immune-stimulating compound is non-covalently linked to the glycogen nanoparticle, for instance via electrostatic interactions, hydrophobic interactions, Van der Waals forces or other non-covalent interactions.

The ratio of immune-stimulating compound covalently or non-covalently linked to the glycogen nanoparticle may be tunable.

The yields of most known methods for producing glycogen or phytoglycogen and most commercial sources are highly polydisperse products that include both glycogen or phytoglycogen particles, as well as other products and degradation products of glycogen or phytoglycogen.

As used herein “glycogen nanoparticles” is used to refer to both glycogen and phytoglycogen nanoparticles, however, it will be understood that in a preferred embodiment, phytoglycogen nanoparticles are used. Accordingly, unless specifically and explicitly excluded, it will be understood the embodiments described include nanoparticles manufactured from plant starting materials.

“Glycogen” can include both products derived from natural sources and synthetic products, including synthetic phytoglycogen i.e. glycogen-like products prepared using enzymatic processes on substrates that include plant-derived material e.g. starch.

In one embodiment, monodisperse glycogen nanoparticles are used. In a further preferred embodiment, monodisperse phytoglycogen nanoparticles are used. In one embodiment, the monodisperse phytoglycogen nanoparticles are prepared according to Example 1. Monodisperse phytoglycogen nanoparticles are commercially available [Phytospherix®, Mirexus Biotechnologies Inc.].

These phytoglycogen nanoparticles are non-toxic, have no known allergenicity, and can be degraded by glycogenolytic enzymes (e.g. amylases and phosphorylases) of the human body. The products of enzymatic degradation are non-toxic molecules of glucose. The production methods described herein enable production of substantially spherical nanoparticles, each of which is a single glycogen molecule.

Glycogen nanoparticles are generally photostable and stable over a wide range of pH, electrolytes, e.g. salt concentrations.

Further. many existing drugs are rapidly eliminated from the body leading to a need for increased dosages. The compact spherical nature of glycogen nanoparticles is associated with efficient cell uptake. while the highly branched nature of glycogen is associated with slow enzymatic degradation, and their high molecular weight (10⁶-10⁷ Da) is believed to be associated with longer intravascular retention time.

Glycogen nanoparticles have properties that address a number of requirements for materials used in pharmaceutical and biomedical applications: predictable biodistribution in different tissues and associated pharmacokinetics; hydrophilicity; biodegradability; and non-toxicity.

United States patent application publication no. United States 20100272639 A1, assigned to the owner of the present application and the disclosure of which is incorporated by reference in its entirety, provides a process for the production of glycogen nanoparticles from bacterial and shell fish biomass. The processes disclosed generally include the steps of mechanical cell disintegration, or by chemical treatment; separation of insoluble cell components by centrifugation; elimination of proteins and nucleic acids from cell lysate by enzymatic treatment followed by dialysis which produces an extract containing crude polysaccharides, lipids, and lipopolysaccharides (LPS) or, alternatively, phenol-water extraction; elimination of LPS by weak acid hydrolysis, or by treatment with salts of multivalent cations, which results in the precipitation of insoluble LPS products; and purification of the glycogen enriched fraction by ultrafiltration and/or size exclusion chromatography; and precipitation of glycogen with a suitable organic solvent or a concentrated glycogen solution can be obtained by ultrafiltration or by ultracentrifugation; and frecze drying to produce a powder of glycogen. Glycogen nanoparticles produced from bacterial biomass were characterized by MWt 5.3-12.7×10⁶ Da. had particle size 35-40 nm in diameter and were monodisperse.

Methods of producing monodisperse compositions of phytoglycogen are described in the International patent application entitled “Phytoglycogen Nanoparticles and Methods of Manufacture Thereof”, published under the international application publication no. WO2014/172786, assigned to the owner of the present application, and the disclosure of which is incorporated by reference in its entirety. In one embodiment. the described methods of producing monodisperse phytoglycogen nanoparticles include: a. immersing disintegrated phytoglycogen-containing plant material in water at a temperature between about 0 and about 50° C.; b. subjecting the product of step (a.) to a solid-liquid separation to obtain an aqueous extract; c. passing the aqueous extract of step (b.) through a microfiltration material having a maximum average pore size of between about 0.05 μm and about 0.15 μm; and d. subjecting the filtrate from step c. to ultrafiltration to remove impurities having a molecular weight of less than about 300 kDa, in one embodiment, less than about 500 kDa, to obtain an aqueous composition comprising monodisperse phytoglycogen nanoparticles. In one embodiment of the method, the phytoglycogen-containing plant material is a cereal or a mixture of cereals, in one embodiment corn. In one embodiment, step c. comprises passing the aqueous extract of step (b.) through (c.1) a first microfiltration material having a maximum average pore size between about 10 μm and about 40 μm; (c.2) a second microfiltration material having a maximum average pore size between about 0.5 μm and about 2.0 μm, and (c.3) a third microfiltration material having a maximum average pore size between about 0.05 and 0.15 μm. The method can further include a step (e.) of subjecting the aqueous composition comprising monodisperse phytoglycogen nanoparticles to enzymatic treatment using amylosucrose, glycosyltransferase, branching enzymes or any combination thereof. The method avoids the use of chemical, enzymatic or thermo treatments that degrade the phytoglycogen material. The aqueous composition can further be dried.

A preferred biomass source is sweet corn (Zea mays var. saccharata and Zea mays var. rugosa), and suitably, the sweet corn is of standard (su) type or sugary enhanced (se) type. In one embodiment, the composition is obtained from dent stage or milk stage kernels of sweet corn. Unlike glycogen from animal or bacterial sources, use of phytoglycogen eliminates the risk of contamination with prions or endotoxins, which could be associated with these other sources.

The polydispersity index (PDI) of a composition of nanoparticles can be determined by the dynamic light scattering (DLS) technique and, in this embodiment, PDI is determined as the square of the ratio of standard deviation to mean diameter (PDI=(σ/d)2. PDI can also be expressed through the distribution of the molecular weight of polymer and, in this embodiment, is defined as the ratio of Mw to Mn, where Mw is the weight-average molar mass and Mn is the number-average molar mass (hereafter this PDI measurement is referred to as PDI*). In the first case, a monodisperse material would have a PDI of zero (0.0) and in the second case the PDI* would be 1.0.

In one embodiment, the pharmaceutical composition comprises monodisperse glycogen nanoparticles having a PDI of less than about 0.3, less than about 0.2, less than about 0.15, less than about 0.10, or less than 0.05 as measured by DLS. In one embodiment, the pharmaceutical composition comprises monodisperse glycogen nanoparticles having a PDI* of less than about 1.3, less than about 1.2, less than about 1.15, less than about 1.10, or less than 1.05 as measured by SEC MALS.

In one embodiment, there is provided a method of sensitizing a cancer cell to a chemotherapeutic compound comprising administering a composition that comprises, consists essentially of, or consists of glycogen-based polysaccharide nanoparticles covalently or non-covalently linked to an immune-stimulating compound concurrently with or within 7 days, preferably within 48 hours of administration of the chemotherapeutic compound.

In various embodiments, the immune-stimulating compound is selected from double-stranded RNA, double-stranded DNA, single-stranded RNA, single stranded DNA, and synthetic analogs thereof. The double-stranded RNA, double-stranded DNA, single-stranded RNA, single stranded DNA, and synthetic analogs thereof are suitably TLR agonists.

In one embodiment, the immune-stimulating compound is polyriboinosinic:polyribocytidilic acid (pI:pC), preferably high molecular weight pI:pC. In one embodiment, the immune-stimulating compound is pI:pC having an average size of >1000 base pairs.

As used herein, “covalently linked” refers to a link via covalent bond, whether directly or via a linker.

As used herein, “non-covalently linked” refers to all non-covalent interactions including electrostatic interactions, hydrophobic interactions, Van der Waals forces and combinations thereof.

In one embodiment, the glycogen-based polysaccharide nanoparticles having an immune-stimulating compound covalently or non-covalently linked thereto have an average particle diameter of between about 10 nm and about 300 nm, in one embodiment, between about 20 nm and 200 nm, in one embodiment about 30 nm to about 150 nm, in one embodiment about 60 nm to about 110 nm, and in other embodiments, about 40 nm to about 140 nm, about 50 nm to about 130 nm, about 60 nm to about 120 nm, about 70 nm to about 110 nm, about 80 nm to about 100 nm, about 10 nm to about 30 nm.

The methods of producing phytoglycogen nanoparticles as detailed in Example 1 and in the international patent application entitled “Phytoglycogen Nanoparticles and Methods of Manufacture Thereof”, are amenable to preparation under pharmaceutical grade conditions.

To impart specific properties to glycogen nanoparticles, they can be chemically modified via numerous methods common for carbohydrate chemistry.

The resulting products are referred to herein as functionalized or modified nanoparticles or derivatives. Functionalization can be carried out on the surface of the nanoparticle, or on both the surface and the interior of the particle, but the structure of the glycogen molecule as a single branched homopolymer is maintained. In one embodiment, the functionalization is carried out on the surface of the nanoparticle. As will be understood by those of skill in the art, chemical modifications should be non-toxic and generally safe for human consumption.

In some embodiments of the present invention, it is advantageous to change the chemical character of glycogen from its hydrophilic, slightly negatively charged native state to be positively charged, or to be partially or highly hydrophobic. J. F Robyt, Essentials of Carbohydrate Chemistry, Springer, 1998; and M. Smith, and J. March, March's Advanced Organic Chemistry: Reactions, Mechanisms, and Structure Advanced Organic Chemistry, Wiley, 2007 provides certain examples of chemical processing of polysaccharides.

Various derivatives can be produced by chemical modification of hydroxyl groups on glycogen, through one or more functionalization steps. Such functional groups include, but are not limited to, nucleophilic and electrophilic groups, acidic and basic groups, e.g., carboxyl groups, amine groups, thiol groups, and aliphatic hydrocarbon groups such as alkyl, vinyl and allyl groups.

In one embodiment, the functionalized nanoparticles are modified with amino groups, which can be primary, secondary, tertiary, or quaternary amino groups, including quaternary ammonium compounds of varying chain lengths. The short-chain quaternary ammonium compound includes at least one alkyl moiety having from 1 to 27 carbon atoms, unsubstituted or substituted with one or more non-carbon heteroatoms (e.g. N, O, S, or halogen).

The nanoparticles described may be functionalized via glycidyltrimethylammonium chloride (GTAC) to render an overall positive charge. In certain embodiments, two or more different chemical compounds are used to produce multifunctional derivatives.

To perform direct conjugation reactions between native glycogen nanoparticles under aqueous conditions, water-soluble chemicals with reactive or activated functionalities (e.g. epoxide or anhydride, pH 8-11) are often necessary. To maintain stability of the glycogen nanoparticles, solution pH is preferably slightly basic, optimally between 8 and 9. As the hydroxyl moieties of the native glucose subunits are not sufficiently reactive (i.e. deprotonated) under these conditions, a significant excess of reagent may be required to obtain appreciable functionalization. Although some reactions (e.g. with alkyl halides) are best conducted in organic solvents such as dimethyl sulfoxide (DMSO) or dimethyl formamide (DMF) to improve reagent solubility and homogeneity, derivatization in aqueous environment is preferable, as these reaction conditions are generally mild and impart low to minimal toxicity.

An alternative chemical modification strategy involves activation of glycogen by appending a functionalized linker or conducting a functional group interconversion of the hydroxyl group, to a more chemically active group. This can be performed in aqueous or organic media, offering the advantage of higher chemical selectivity and efficiency. It is possible to isolate the activated glycogen precursors (e.g. aminated, carboxylated) which can then be coupled with a suitable reagent. As detailed in the Examples, the present inventors have synthesized a number of nanoparticle functional derivatives using this method. Nucleophilic and electrophilic groups (such as amino or hydrazide and aldehyde groups, respectively) have been attached to the glycogen nanoparticle backbone.

By way of example, the simplest approach is the introduction of carbonyl groups by selective oxidation of glucose hydroxyl groups at positions of C-2, C-3, C-4 and/or C-6. There is a wide spectrum of redox agents which can be employed, such as persulfate, periodate, bromine, acetic anhydride, Dess-Martin periodinane, TEMPO (2,2,6,6-Tetramethylpiperidin-1-yl)oxyl), etc.

Glycogen nanoparticles functionalized with carboxylate groups are readily reactive towards compounds bearing primary or secondary amine groups. The coupling of these two partners (e.g. through EDC coupling chemistry) results in the formation of amides. This chemistry could also be employed in the reverse direction: reacting amine functionalized glycogen with carboxylate-containing compounds.

For the preparation of primary, secondary or tertiary amino-functionalized nanoparticles, one method of the current invention utilizes the reaction of native glycogen with 2-aminoalkyl halides or hydrogen sulfate. Treatment of the glycogen under basic (pH 9-12) conditions with aminoalkyl substrates results in a nucleophilic substitution reaction, displacing the halide or hydrogen sulfate leaving group. As a result, the glycogen is aminoalkylated (e.g. primary, secondary, or tertiary aminated with an O-alkyl linker). The reaction can be performed at a variety of temperatures (25-90° C.) and aminoalkylating agents of varying chain lengths or leaving groups.

For the quaternary ammonium modification of glycogen, the native nanoparticles are reacted with a variety of 3-chloro-2-hydroxypropyltrialkylammonium chloride reagents, which exist in an epoxide-chlorohydrin equilibrium, depending on solution pH. Under the basic conditions of the reaction performed herein (pH 9-12), the quaternary ammonium reagents are in the epoxide form, which react readily by base-catalyzed ring-opening with the glycogen. The resulting products are 3-(trimethylammonio)-2-hydroxyprop-1-yl or 3-(N-alkyl-N,N-dimethylammonio)-2-hydroxyprop-1-yl glycogen, where in the latter case, the alkyl groups are long-chain alkyl groups including lauryl (C12), cocoalkyl, (C8-C18), and stearyl (C12-C27).

The above quaternary ammonium modified glycogen can then be further reacted with various alkyl, benzyl, or silyl halides to afford nanoparticles bearing both hydrophilic (cationic, quaternary ammonium) and hydrophobic functionalities.

Another route to primary amination of glycogen includes a two-step sequence involving imides. Native glycogen is first reacted under basic conditions with an imide-containing epoxide or alkyl halide, by the chemistry described above, to provide the corresponding (N-imidyl) protected aminoalkyl glycogen ( . . . Eq. 1). Depending on the nature of the imide reagent used, the length of the O-alkyl tether and substituents (e.g. imide bearing various alkyl/aryl cyclic or acyclic groups) may be tailored. The N-imidyl group on this product can then be removed by one of several conditions (reducing agent followed by acetic acid at pH 5, aqueous hydrazine hydrate, methylamine, etc.) to afford primary aminoalkylated glycogen nanoparticles (Eq. 2).

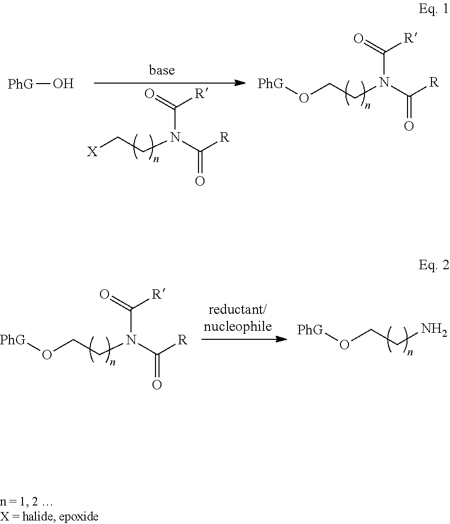

Reductive amination of the nanoparticles can be also achieved by the following two step process. First step is cyanoalkylation, i.e., converting hydroxyls into O-cyanoalkyl groups by reaction with bromoacetonitrile or acrylonitrile. In the second step, the cyano groups are reduced with metal hydrides (borane-THF complex, LiAlH₄, etc).

Amino-functionalized nanoparticles are amenable to further modifications. Amino groups are reactive to carbonyl compounds (aldehydes and ketones), carboxylic acids and their derivatives, (e.g. acyl chlorides, esters), succinimidyl esters, isothiocyanates, sulfonyl chlorides, etc.

In one embodiment, the immune-stimulating compound is covalently linked to the glycogen-based polysaccharide nanoparticles. In one embodiment, glycogen-based polysaccharide nanoparticles are linked directly via covalent bond to an immune-stimulating compound.

In another embodiment, the glycogen-based polysaccharide nanoparticles are cationized and the immune-stimulating compound is linked to the cationized glycogen-based polysaccharide nanoparticles via non-covalent interactions, in one embodiment ionic bonding.

A chemical compound bearing a functional group capable of binding to carbonyl-, cyanate-, imidocarbonate or amino-groups can be directly attached to functionalized glycogen nanoparticles. Chemical compounds may also be attached via a polymer spacer or a “linker”. These can be homo-or hetero-bifunctional linkers bearing functional groups such as amino, carbonyl, sulfhydryl, succimidyl, maleimidyl, and isocyanate, (e.g. diaminohexane), ethylene glycobis (sulfosuccimidylsuccinate), disulfosuccimidyl tartarate, dithiobis (sulfosuccimidylpropionate), aminoethanethiol, etc.

Suitable methods of covalently bonding the immune-stimulating compound to the glycogen-based polysaccharide nanoparticles are described above and in the examples. An example method is provided in Example 5.

Suitable methods for cationizing glycogen nanoparticles enabling a non-covalent complex between the glycogen nanoparticles and the immune-stimulating compounds are described above and in the examples. An example method is provided in Example 6.

The nanoparticles may also be covalently linked directly or via a spacer, to one or more compounds such diagnostic labels (which may have light absorbing, light emitting, fluorescent, luminescent, Raman scattering, fluorescence resonant energy transfer, and electroluminescence properties), chelating agents, dispersants, surfactants, charge modifying agents, viscosity modifying agents, hydrophobicity modifiers, coagulation agents and flocculants, as well as various combinations of the above.

In one embodiment, the glycogen-based polysaccharide nanoparticles are further linked, directly or via a spacer, to one or more small molecules, proteins, peptides, antibodies or antibody fragments, aptamers, receptor ligands, for directing the nanoparticles to a specific cell type or cellular compartment.

For example, and without limiting the generality of the foregoing, glycogen-based polysaccharide nanoparticles may be covalently linked to at least one immune-stimulating compound and may further be covalently linked to a diagnostic or targeting label. In another example, cationized glycogen nanoparticles may be non-covalently linked with an immune-stimulating compound and the nanoparticles may be further covalently linked to a diagnostic or targeting label.

In one embodiment the immune-stimulating compound is a macromolecule, suitably, a nucleic acid.

In one embodiment, the size of the nucleic acid is not particularly restricted. The nanoparticles described herein can be covalently or non-covalently linked to nucleic acids having ≥10,000 base pairs. In one embodiment, the nucleic acid is between 10 and 10,000 nucleotides in length. In one embodiment, between 1000 and 10,000 nucleotides in length.

The immune-stimulating compound may suitably be a double-stranded (ds) RNA, dsDNA or single-stranded (ss) RNA, ssDNA, or a synthetic analog of any of the foregoing (e.g. poly IC, CpG DNA, CpG ODNs, LNAs).

In one embodiment, the immune-stimulating compound is a synthetic (ds)RNA; in one embodiment polyinosinic: polycytidilic acid (pI:pC).

The immune-stimulating compounds, covalently or non-covalently linked to glycogen-based polysaccharide nanoparticles, as described herein provide a surprising and unexpected magnitude of induction or enhancement of an immune response in cancer cells.

The combination of properties of glycogen nanoparticles described above together with the feasibility of low production costs, makes the nanoparticle compositions described herein highly suitable for the uses as described herein.

As demonstrated in the Examples, glycogen nanoparticles can carry molecules that enhance an innate immune response across the cancer cell membrane, such molecules being effective within the cells to enhance an immune response and sensitizing the cancer cells to treatment with a chemotherapeutic compound.

In one embodiment, there is provided a method comprising administering a therapeutically effective amount of a pharmaceutical composition comprising an immune-stimulating compound covalently or non-covalently linked to a glycogen-based nanoparticle to a cancer patient.

As used herein “patient” refers to an organism being treated, in one embodiment a vertebrate organism, in one embodiment a mammal, in one embodiment a human patient.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

Each glycogen particle is a single molecule, made of highly-branched glucose homopolymer characterized by very high molecular weight (up to 10⁷ Da). This homopolymer consists of α-D-glucose chains with 1→4 linkage and branching points occurring at 1→6 and with branching degree about 10%. These particles are spherical and can be manufactured with different sizes by varying the starting material and filtering steps.

Glycogen nanoparticles are water soluble and by attaching compounds to glycogen nanoparticles they can be made water soluble. The high density of surface groups on the glycogen particles results in a variety of unique properties of glycogen nanoparticles, such as fast dissolution in water, low viscosity and shear thinning effects for aqueous solutions at high concentrations of glycogen nanoparticles. This is in contrast to high viscosity and poor solubility of linear and low-branched polysaccharides of comparable molecular weight. Furthermore, it allows formulation of highly concentrated (up to 30%) stable dispersions in water or DMSO.

The novel formulations of the invention may also be admixed, encapsulated, or otherwise associated with other molecules, molecule structures or mixtures of compounds and may be combined with any pharmaceutically acceptable carrier or excipient. As used herein, a “pharmaceutically acceptable carrier” or “excipient” can be a pharmaceutically acceptable solvent, suspending agent or any other pharmacologically inert vehicle for delivering functionalized glycogen nanoparticles, covalently or non-covalently linked to an immune-stimulating compound, to an animal. The excipient may be liquid or solid and is selected with the planned manner of administration in mind, so as to provide for the desired bulk, consistency, etc., when combined with glycogen nanoparticles and the other components of a given pharmaceutical composition. Examples of pharmaceutically acceptable carriers include one or more of water, saline, phosphate buffered saline, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the pharmacological agent.

In an embodiment, the compositions may be lyophilized or spray dried and may be subsequently formulated for administration. For example, in the case of a composition of a nucleic acid immune-stimulating compound non-covalently linked to glycogen nanoparticles, the composition can be lyophilized or spray dried, yielding a product that is stable under storage/transport conditions that would not require cold chain (i.e. no need for refrigeration).

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery).

For the purposes of formulating pharmaceutical compositions, monodisperse glycogen nanoparticles prepared as taught herein, may be provided in a dried particulate/powder form or may be dissolved e.g. in an aqueous solution. Where a low viscosity is desired, the glycogen nanoparticles may suitably be used in formulations in a concentration of up to about 25% w/w. In applications where a high viscosity is desirable, the nanoparticles may be used in formulations in concentrations above about 25% w/w. In applications where a gel or semi-solid is desirable, concentrations up to about 35% w/w can be used, or the nanoparticles can be used in combination with viscosity builders or gelling agents.

The pharmaceutical compositions of the present invention may be administered in a number of ways depending upon whether local or systemic treatment is desired and upon the area to be treated.

Without limiting the generality of the foregoing, compositions may be administered intravenously, interperitoneally, orally, intratumourally, intramuscularly or subcutaneously.

### EXAMPLES

### Manufacture of Phytoglycogen from Sweet Corn Kernels

1 kg of frozen sweet corn kernels (75% moisture content) was mixed with 2 L of deionized water at 20° C. and was pulverized in a blender at 3000 rpm for 3 min. Mush was centrifuged at 12,000×g for 15 min at 4° C. The combined supernatant fraction was subjected to cross flow filtration (CFF) using a membrane filter with 0.1 μm pore size. The filtrate was further purified by a batch diafiltration using membrane with MWCO of 500 kDa and at RT and diavolume of 6. (Diavolume is the ratio of total mQ water volume introduced to the operation during diafiltration to retentate volume.)

The retentate fraction was mixed with 2.5 volumes of 95% ethanol and centrifuged at 8,000×g for 10 min at 4° C. The retentate was mixed with 2.5 volumes of 95% ethanol and centrifuged at 8,000×g for 10 min at 4° C. The pellet containing phytoglycogen was dried in an oven at 50° C. for 24 h and then milled to 45 mesh. The weight of the dried phytoglycogen was 97 g.

According to dynamic light scattering (DLS) measurements, the phytoglycogen nanoparticles produced had particle size diameter of 83.0 nm and a polydispersity index of 0.081.

### Quaternary Ammonium Cationization of Phytoglycogen

Phytoglycogen nanoparticles (PhG) are mixed with aqueous sodium hydroxide solution (1.5-60.0 mmol of NaOH dissolved in 1-5 ml of water/g PhG) and heated to 25 or 45° C. Over the course of 10-120minutes, 2,3-epoxypropyltrimethylammonium chloride in water (69% solution, 3.07 mL/g PhG) is added. Alternatively, 8.23 mmol of (3-chloro-2-hydroxy-prop-1-yl) dimethylalkyl ammonium chloride (alkyl=lauryl, cocoalkyl, stearyl) is stirred with 0.82 mL of 50% NaOH at 45° C. for 5 minutes, and 3.33 ml of 20% aqueous solution of PhG is added. Each reaction is stirred for another 2-6 hours at 45° C. Water (5-9 mL/g PhG) is then added, the mixture is cooled to room temperature and neutralized with 1 M HCl. The product is precipitated and washed in ethanol or hexanes (50-80 mL/g PhG), re-dissolved in water (20 mL/g PhG) and/or saturated NaCl (10 mL) and further purified by dialysis. Freeze-drying affords the product as a white solid. DS (NMR): 0.14-1.46 (Table 3).

### Alkylation, Benzylation, or Silylation of Trimethylammonium-Cationized Phytoglycogen

Trimethylammonium-cationized PhG from Example 2 is oven-dried at 105° C. for 16 h (silylation) or used as is (alkylation/benzylation). The PhG was dissolved in dry dimethylsulfoxide (20 mL/g PhG) at 80° C. for 1 hour. For alkyl/benzylation reactions, water (0.5 mL) and 50% NaOH (0.042-2.47 mmol/g PhG) is added and stirred vigorously for 10 minutes. Alkyl or benzyl halides (0.51-30.6 mmol/g glycogen) are then added and the mixture is stirred for 2 hours at 60° C., cooled to room temperature and neutralized with glacial acetic acid. For benzylation, the addition of benzyl bromide (0.51 mmol/g PhG) could be performed at 60° C. for 2 hours directly following cationization at 45° C. for 2-6 hours, as a one-pot synthesis. For silylation, the reaction vessel is capped with a rubber septum, cooled to 0° C., and triethylamine (1.19-4.75 mmol/g PhG) is added, followed by dropwise addition of silyl chloride (trimethylsilyl chloride (0.36-1.46 mmol/g PhG), triethylsilyl chloride (0.36 mmol/g PhG)) and stirred overnight at room temperature. For alkylation/benzylation, the crude mixture is extracted several times with diethyl ether/hexanes/ethanol and re-suspended in saturated aqueous NaCl of pH 5-7, then dialysed and freeze-dried; whereas for silylation, the product is precipitated into acetonitrile and washed with hot acetonitrile, then dried overnight at 60° C. and 100 mbar. All are white solids. DS (NMR): alkylation 0.004-1.58; one-pot benzylation: 0.068 for benzyl group and 1.12 for cationic group; silylation 0.19-0.45 (Table 1).

TABLE 1
Reaction parameters and characterization data for Example 2 and Example 3.

| | | Alkyl | Cationizing | | | |
|---|---|---|---|---|---|---|
| | NaOH | halide | agent | | Hydrodynamic | |
| | (mmol/g | (mmol/g | (mmol/g | | diameter | ζ-potential |
| Substituent | PhG) | PhG) | PhG) | DS_{NMR} | (DLS) | (mV) |
| Ethyl | 0.04 | 0.51 | — | 0.006 | 61.95 | 53.3 |
| Ethyl | 0.17 | 2.04 | — | 0.083 | 64.81 | 51.4 |
| Ethyl | 0.41 | 5.10 | — | 0.66 | 64.38 | 51.3 |
| Dodecyl | 0.04 | 0.51 | — | 0.004 | 76.91 | 57.1 |
| Dodecyl | 0.17 | 2.04 | — | 0.17 | 166 | 33.3 |
| Benzyl | 0.04 | 0.51 | — | 0.052 | 64.57 | 57.9 |
| Benzyl | 0.17 | 2.04 | — | 0.528 | 70.19 | 57.5 |
| Benzyl | 0.41 | 5.10 | — | 1.2 | 78.64 | 62.5 |
| Benzyl | 0.99 | 12.24 | — | 1.58 | 65.61 | 51.6 |
| Benzyl/QUAB151 | 2.5 | 0.51 | 12.4 | 0.068 | 49.98 | 39.9 |
| QUAB 151 | 2.35 | — | 0.28 | 0.34 | 63.81 | 37.4 |
| QUAB 151 | 2.35 | — | 0.56 | 0.47 | 59.18 | 37.6 |
| QUAB 342 | 15.5 | — | 12.5 | 0.88 | 62.74 | 67.7 |
| QUAB 360 | 15.5 | — | 12.5 | 1.02 | 76.58 | 52.6 |
| QUAB 426 | 15.5 | — | 12.5 | 0.136 | 165.7 | 57.7 |

Abbreviations: QUAB, (3-chloro-2-hydroxy-prop-1-yl)dimethylalkyl ammonium chloride (alkyl = lauryl (342), cocoalkyl (360), stearyl (426)), QUAB 151: (3-chloro-2-hydroxy-prop-1-yl)trimethylammonium chloride.

### TEMPO Oxidized Phytoglycogen

Phytoglycogen nanoparticles (PhG) are dissolved in glycine buffer (0.05 M, pH 10.20, 33 mL/g PhG). The sample is placed in an ice bath. After 4 hours, a solution of TEMPO (0.04 g/g PhG) in glycine buffer (1.7 mL/g PhG) is added to the reaction mixture. NaBr (0.60 g/g PhG) is then added. After an hour (reaction at 3° C.), NaClO solution (4.52% chlorine) is introduced over the course of 30 minutes (80 mL/g PhG per addition). The reaction mixture is stirred at 0-5° C. for 72 hours, then quenched with anhydrous ethanol (13 mL/g PhG). The mixture is dialyzed (12-14 kDa cut-off) against RO water for 6 cycles, and lyophilized to afford an off-white powder.

### Poly IC Bound Phytoglycogen

All solutions and glassware were sterile or autoclaved (121° C. for 30 minutes) and nuclease-free where possible. 1.3 mg of native PhG (prepared as in Example 1) or TEMPO-oxidized PhG (Example 4) is dissolved in 0.5 M MES solution, pH 6.4 (0.5 mL). In a separate vial, poly IC (polyinosinic: polycytidylic acid, 4.2 mg) is dissolved in PBS solution (0.5 mL). The PhG and poly IC solutions were stirred at 50° C. for 30 minutes. In a third vial, MES solution at room temperature (3.4 mL) was set aside and 0.1 mL of neat EDC (N-(3-dimethylaminopropyl)-N′-ethylcarbodiimide) was added, and the pH was adjusted to 6.7 with NaOH. 1 mL of this EDC/MES solution was added to poly IC-PBS solution, which was transferred into PhG-MES solution. Two controls were also prepared: one without Poly IC, and one without EDC. All reactions were left to stir at 50° C. for 1 hour, then cooled to room temperature. Each reaction mixture was transferred into 100K centrifugal filtration devices (3×0.5 mL) and spun down at 15,000×g for 10 minutes to recover the retentate. Alternatively, samples were dialyzed (MWCO 12-15,000 Da against RO water) for 2 days and lyophilized, or precipitated in ethanol (20 mL) then pelleted by centrifugation (15 min at 7500×g) and dried at 25-50° C. for 6-24 hours.

### Binding of Poly IC by Cationic PhG

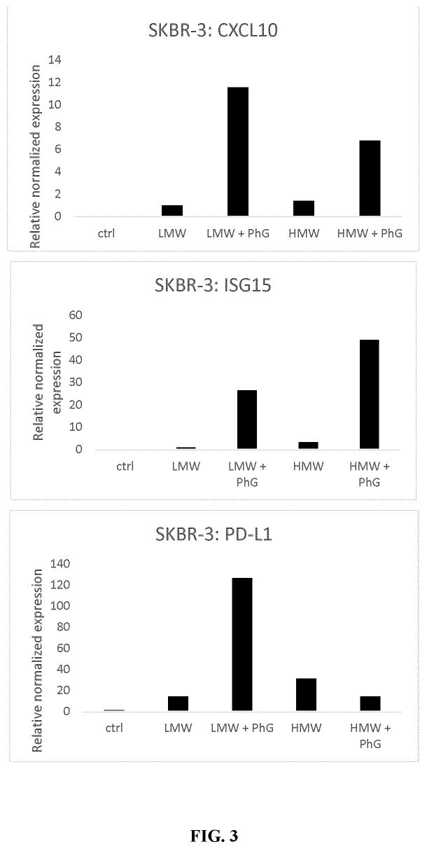

The ability of cationic phytoglycogen nanoparticles (prepared according to Example 5 Table 1) (Cat-PhG) to bind poly IC was demonstrated by electrophoretic mobility shift assay (EMSA). Three (3) micrograms of poly IC was mixed with 2-fold dilution series of Cat-PhG with DS 0.88 (Example 2). After a 20-minute incubation at room temperature, samples were separated on a 1% agarose gel, stained with ethidium bromide, and subsequently imaged under UV light. Shown in FIG. 3 are the EMSA gels for Cat-PhG-0.88 (Example 5) demonstrating maximum loading ˜6:1 (poly IC:PhG w/w).

### Example 7. Binding of pI:pC to Cationic PhG for Treatment of Cells

For preparation of glycogen nanoparticles with pI:pC, a stock solution of pI:pC (1 mg/ml) was heated to 55° C. for 10 minutes, mixed by repeated pipetting, heated for an additional 10 minutes at 55° C., then diluted to 10 μg/ml in molecular grade water. Cationic glycogen was dispersed in molecular grade water at 1 mg/ml then diluted to 200 μg/ml in molecular grade water. An appropriate amount of glycogen nanoparticle was dispensed into a fresh tube, then an appropriate amount of pI:pC was added and the solution was immediately mixed by repeated pipetting, and allowed to incubate at room temperature for 20minutes. For example, to prepare 3 μg of pI:pC with 6 μg of glycogen nanoparticle, 30 μl of 200 μg/ml nanoparticle would be complexed with 300 μl of 10 μg/ml solution of pI:pC.

### Example 8. PhG Complexed with pI:pC Induces Robust Innate Immune Response in Cancer Cells

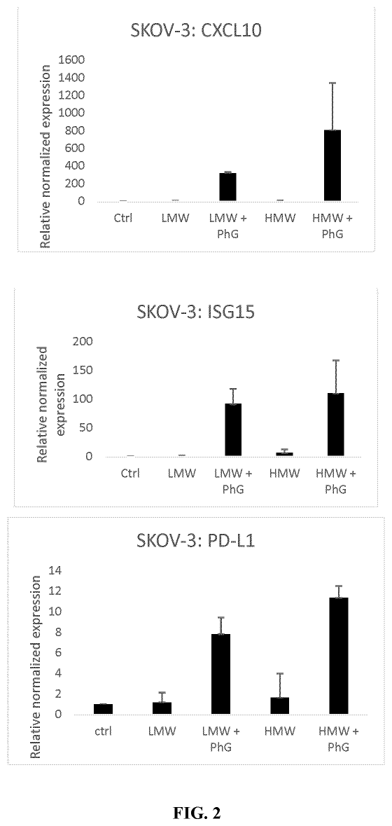

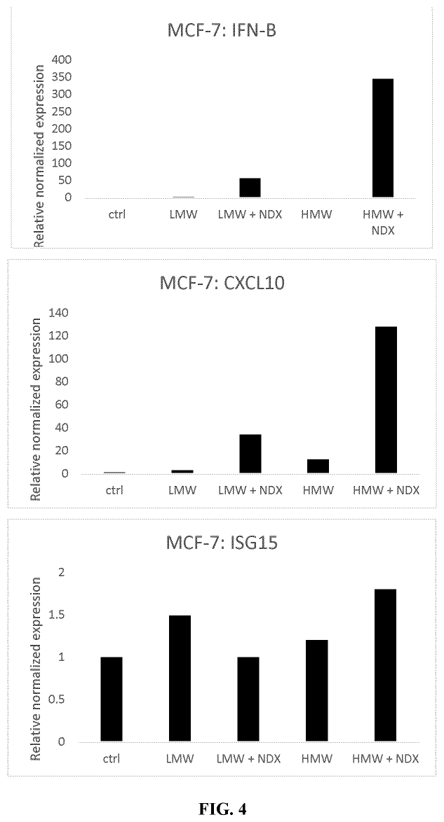

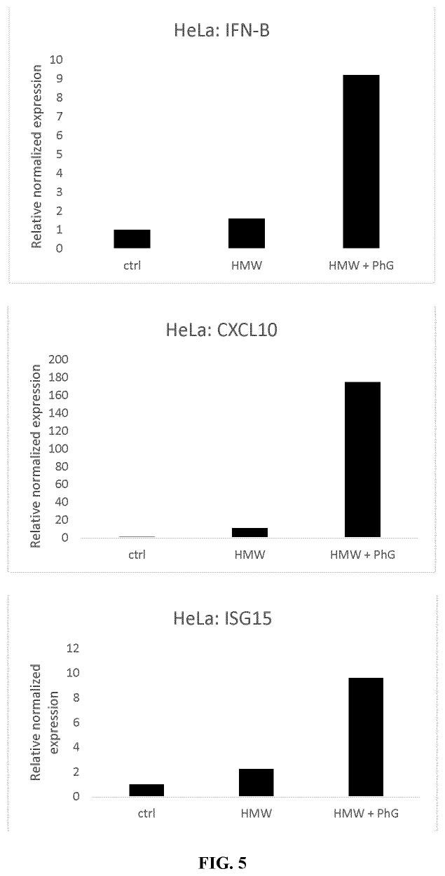

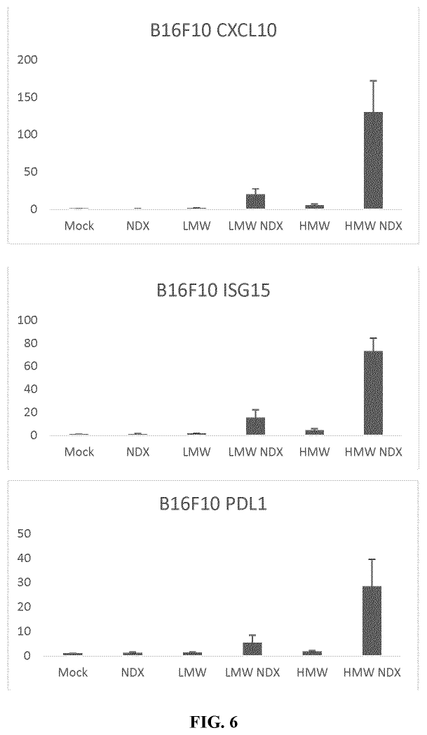

FIGS. 2-6 demonstrate stimulation of the innate immune response in several cancer cell lines; SKOV3 (Ovarian cancer), SKBR3 and MCF7 (Breast cancer), HeLa (Cervical cancer), B16F10(Melanoma). Cells were plated in 6-well plates at 1×10E6 cells per well in DMEM and allowed to attach overnight. After attachment cell were mock treated or treated with either “free” low molecular weight (LMW) or high molecular weight (HMW) pI:pC, or LMW or HMW pI:pC bound to cationic PhG at a 2:1w/w ratio (PhG:pI:pC). After a 6-hour incubation at 37° C., total RNA was isolated via Trizol reagent (Thermo-Fisher). Subsequently, 500 ng of total RNA was converted to cDNA using iScript (BioRad); cDNA was diluted 1:10 and 2 μl was used in a qPCR reaction (EvaGreen, BioRad) with primers specific for human actin, IFN-beta, ISG15, or CXCL10 (Table 2). All transcripts were normalized to actin and quantified relative to mock treated. Cells treated with Cat-PhG-poly IC demonstrated greater transcript levels of all interferon-stimulated genes (ISGs) assayed compared to both mock and “free” poly IC treated cells in all cancer cell lines tested.

TABLE 2
Primers designed for qPCR experiments

| Target | Forward primer (5′-3′) | Reverse Primer (5′-3′) |
|---|---|---|
| Human isg15 | cagccatgggctgggac (SEQ ID | cttcagctctgacaccgaca (SEQ ID |
| | NO: 1) | NO: 2) |
| Human interferon | aaactcatgagcagtctgca (SEQ ID | aggagatcttcagtttcggagg (SEQ ID |
| beta | NO: 3) | NO: 4) |
| Human cxcl10 | gaaagcagttagcaaggaaagg (SEQ | gacatatactccatgtagggaagtg (SEQ |
| | ID NO: 5) | ID NO: 6) |
| Human PDL1 | ggagattagatcctgaggaaaacca | aacggaagatgaatctcagtgcta (SEQ |
| | (SEQ ID NO: 7) | ID NO: 8) |
| Human beta actin | ctggcacccagcacaatg (SEQ ID | ccgatccacacggagtacttg (SEQ ID |
| | NO: 9) | NO: 10) |

### Example 9. Cell Viability of pI:pC Resistant SKOV-3 Cells Treated with Carboplatin Alone or in Combination with “Free” pI:pC or Glycogen Nanoparticle Bound pI:pC

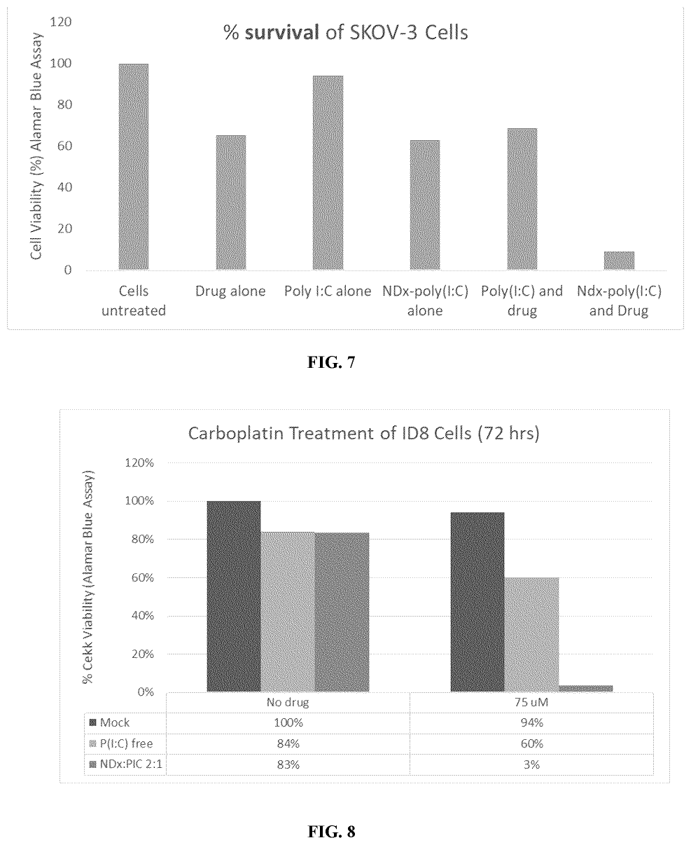

Cell viability was measured by alamar blue of pI:pC resistant SKOV-3 cells treated with carboplatin alone, or in combination with “free” pI:pC or glycogen nanoparticle bound pI:pC. SKOV-3 cells were plated in a 12-well dish at 1.5×10⁶ per well. Twenty-four (24) hours post seeding, cells were left untreated, or treated with EC50 carboplatin, 125 ng/ml pI:pC, 125 ng/ml pI:pC ionically bound to glycogen nanoparticles (1:1 w/w), or the combination of EC50 carboplatin and 125 ng/ml pI:pC, or EC50carboplatin and 125 ng/ml nanoparticle bound pI:pC. Cells were incubated at 37° C., 5% CO₂ for 72 hours, and cell viability was measured by alamar blue assay according to the manufacturer's protocol (Invitrogen). Results are shown in FIG. 7.

### Example 10. Cell Viability of pI:pC Resistant ID8 Cells Treated with Carboplatin Alone or in Combination with “Free” pI:pC or Glycogen Nanoparticle Bound pI:pC

Cell viability was measured by alamar blue of ID8 murine ovarian cancer cells treated with carboplatin alone, or in combination with “free” pI:pC or glycogen nanoparticle bound pI:pC. ID8 cells were plated in a 96-well dish at 1.5×10⁶ ml. Twenty-four (24) hours post seeding, cells were left untreated, or treated with 75 μM carboplatin, 5 μg/ml pI:pC, 5 μg/ml nanoparticle bound pI:pC (2:1 w/w), or the combination of 75 μM carboplatin and 5 μg/ml pI:pC, or 75 μM carboplatin and 5 μg/ml nanoparticle bound pI:pC. Cells were incubated at 37° C., 5% CO₂ for 72 hours, and cell viability was measured by alamar blue assay according to the manufacturer's protocol (Invitrogen). Results are shown in FIG. 8.

This application includes a Sequence Listing which has been submitted in ASCII format and herby incorporated by reference in its entirety. Said ASCII copy was created on Feb. 20, 2024 and is named SL_095576-00007.txt and has a size of 1761 bytes.

### REFERENCES

1) Critchley-Thorne R J, Simons D L, Yan N, et al. Impaired interferon signaling is a common immune defect in human cancer. Proc Natl Acad Sci U S A. 2009;106(22):9010-9015. doi: 10.1073/pnas.0901329106
2) Van D N, Roberts C F, Marion J D, et al. Innate immune agonist, dsRNA, induces apoptosis in ovarian cancer cells and enhances the potency of cytotoxic chemotherapeutics. FASEB J. 2012;26(8):3188-3198. doi: 10.1096/fj.11-202333
3) Ding L, Ren J, Zhang D, et al. The TLR3 Agonist Inhibit Drug Efflux and Sequentially Consolidates Low-Dose Cisplatin-Based Chemoimmunotherapy while Reducing Side Effects. Mol Cancer Ther. 2017;16(6):1068-1079. doi: 10.1158/1535-7163.MCT-16-0454
4) Stolfi R L, Martin D S. Modulation of chemotherapeutic drug activity with polyribonucleotides or with interferon. J Biol Response Mod. 1985;4(6):634-639.
5) Le U M, Yanasarn N, Löhr C V, Fischer K A, Cui Z. Tumor chemo-immunotherapy using gemcitabine and a synthetic dsRNA. Cancer Biol Ther. 2008; 7(3):440-447. doi: 10.4161/cbt.7.3.5423
6) Taura M, Fukuda R, Suico M A, et al. TLR3 induction by anticancer drugs potentiates poly I:C-induced tumor cell apoptosis. Cancer Sci. 2010;101(7):1610-1617. doi: 10.1111/j.1349-7006.2010.01567.x
7) Monlish D A, Bhatt S T, Schuettpelz L G. The Role of Toll-Like Receptors in Hematopoietic Malignancies. Front Immunol. 2016;7:390. Published 2016 Sep. 28. doi: 10.3389/fimmu.2016.00390

What is claimed is:

1. A method of sensitizing a cancer cell to a chemotherapeutic drug comprising contacting the cell with an immune-stimulating compound covalently or non-covalently linked to glycogen-based polysaccharide nanoparticles having a molecular weight of 10⁶ to 10⁷ Da comprising α-D glucose chains, having an average chain length of 11-12, with 1→4 linkage and branching point occurring at 1→6 and with a branching degree of between 6% and 13%.

2. The method of claim 1, wherein sensitizing a cancer cell to a chemotherapeutic drug comprises stimulating an immune response in the cancer cell, which comprises increasing expression of one or more gene(s) associated with the innate immune response, apoptosis and/or autophagy.

3. The method of claim 2, wherein stimulating an immune response comprises increasing expression of one or more of IFN-beta, ISG-15 and CXCL10.

4. The method of any one of claims 1 to 3, wherein sensitizing cancer cells to a chemotherapeutic drug comprises lowering the EC50 by >10%, >20%, >30%, >40%, >50%, >60%, >70%, >80%, or >90%.

5. The method of any one of claims 1 to 4, wherein the a chemotherapeutic drug comprises a platinum-based compound, an anthracycline (including doxorubicin, daunorubicin, epirubicin, idarubicin), etoposide, 2′, 2′-difluoro 2′deoxycytidine (dFdC, gemcitabine), capecitabine, fluorouracil, a taxane (including Paclitaxel, Docetaxel), irinotecan, methotrexate, pemetrexed, topotecan, vinblastine, vincristine, auristatin E, auristatin F, dolastatin, maytansinol, monomethyl auristatin E, monomethyl auristatin F, monomethyl auristatin D, DM1, DM4, Pyrrolobenzodiazepine, alpha-aminitin, calicheamicin, campathecin, and/or mitomycin C.

6. The method of claim 5, wherein the a chemotherapeutic drug comprises a platinum-based chemotherapeutic compound, preferably selected from carboplatin, cisplatin and oxaliplatin.

7. The method of claim 1, wherein sensitizing the cancer cell to a chemotherapeutic drug comprises inducing expression of PDL-1 in the cancer cell and wherein the chemotherapeutic drug comprises a checkpoint inhibitor.

8. The method of any one of claims 1 to 7, wherein the immune-stimulating compound is a TLR agonist, preferably selected from: double-stranded RNA, double-stranded DNA and single-stranded RNA, single-stranded DNA, or any synthetic analogs thereof including poly IC, CpG ODN, LNA.

9. The method of claim 8, wherein the immune-stimulating compound is poly IC.

10. The method of any one of claims 1 to 9 wherein the immune-stimulating compound is covalently linked to the nanoparticles through a linking group.

11. The method of any one of claims 1 to 9 wherein the nanoparticles are cationic and the immune-stimulating compound is non-covalently linked to the nanoparticles through electrostatic interactions.

12. The method of any one of claims 1 to 11, wherein the immune-stimulating compound comprises between % 50-%600 by weight relative to the glycogen-based polysaccharide nanoparticles.

13. The method of any one of claims 1 to 11 wherein the glycogen-based polysaccharide nanoparticles and the immune-stimulating compound are used in a weight ratio of 2:1.

14. The method of any one of claims 1 to 13, wherein the glycogen-based polysaccharide nanoparticles have a polydispersity index as measured by DLS of less than 0.3 and the glycogen-based polysaccharide nanoparticles linked to the immune-stimulating compound have an average particle diameter of between about 10 nm and about 300 nm.

15. The method of any one of claims 1 to 14 comprising contacting the cell with the chemotherapeutic drug within ±7 days, within ±48 hours, within ±24 hours or concurrently with contacting the cell with the immune-stimulating compound covalently or non-covalently linked to glycogen-based polysaccharide nanoparticles.

16. The method of any one of claims 1 to 15 wherein the cell is in a cancer patient and the method comprises administering a therapeutically effective amount of a pharmaceutical composition comprising the immune-stimulating compound covalently or non-covalently linked to the glycogen-based polysaccharide nanoparticles to the cancer patient.

17. The method of claim 16, wherein the nanoparticles are further covalently linked to one or more small molecules, proteins, peptides, antibodies or antibody fragments, aptamers, receptor ligands, for directing the nanoparticles to a specific cell type or cellular compartment.

18. The method of any one of claims 16 to 17, comprising administering the immune-stimulating compound covalently or non-covalently linked to glycogen-based polysaccharide nanoparticles within 7 days, preferably within 48 hours of administering the chemotherapeutic drug.

19. The method of any one of claims 1 to 17, comprising administering the immune-stimulating compound covalently or non-covalently linked to glycogen-based polysaccharide nanoparticles concurrently with the chemotherapeutic drug.

20. The method of any one of claims 16 to 19, wherein the patient is diagnosed with glioma, thyroid cancer, lung cancer, colorectal cancer, stomach cancer, liver cancer, carcinoid, pancreatic, renal, urothelial, prostate, breast, cervical, endometrial, ovarian, melanoma, multiple myeloma, acute myeloid leukemia, indolent non-Hodgkin's lymphoma.

21. The method of any one of claims 16 to 20, wherein the patient is diagnosed with ovarian cancer or breast cancer.


SEQUENCE LISTING (10 sequences)

SEQ ID NO: 1 - DNA, 17 bp, Artificial Sequence
  primer sequence
cagccatgggctgggac

SEQ ID NO: 2 - DNA, 20 bp, Artificial Sequence
  primer sequence
cttcagctctgacaccgaca

SEQ ID NO: 3 - DNA, 20 bp, Artificial Sequence
  primer sequence
aaactcatgagcagtctgca

SEQ ID NO: 4 - DNA, 22 bp, Artificial Sequence
  primer sequence
aggagatcttcagtttcggagg

SEQ ID NO: 5 - DNA, 22 bp, Artificial Sequence
  primer sequence
gaaagcagttagcaaggaaagg

SEQ ID NO: 6 - DNA, 25 bp, Artificial Sequence
  primer sequence
gacatatactccatgtagggaagtg

SEQ ID NO: 7 - DNA, 25 bp, Artificial Sequence
  primer sequence
ggagattagatcctgaggaaaacca

SEQ ID NO: 8 - DNA, 24 bp, Artificial Sequence
  primer sequence
aacggaagatgaatctcagtgcta

SEQ ID NO: 9 - DNA, 18 bp, Artificial Sequence
  primer sequence
ctggcacccagcacaatg

SEQ ID NO: 10 - DNA, 21 bp, Artificial Sequence
  primer sequence
ccgatccacacggagtacttg